(19) United States
(12) Patent Application Publication
(10) Pub. No.: US 2015/0246131 A1
(43) Pub. Date: Sep. 3, 2015

(54) WATER-BASED PERSONAL LUBRICANTS WITH A SILICONE COMPONENT

(71) Applicant: Church & Dwight, Co., Inc., Princeton, NJ (US)

(72) Inventors: Ovidiu Romanoschi, Highland Park, NJ (US); Luis Muniz, Brooklyn, NY (US); Michael J. Harrison, Princeton, NJ (US); Jason Kieke, Freehold, NJ (US)

(73) Assignee: Church & Dwight, Co., Inc., Princeton, NJ (US)

(21) Appl. No.: 14/428,938

(22) PCT Filed: Oct. 2, 2013
(86) PCT No.: PCT/US13/63029

§ 371 Date: Mar. 17, 2015

Related U.S. Application Data
(60) Provisional application No. 61/709,490, filed on Oct. 4, 2012.

(51) Int. Cl. A61K 47/34; A61K 47/10; A61K 9/06; A61K 9/00

(52) U.S. Cl. CPC A61K 47/34; A61K 9/0034; A61K 47/10; A61K 9/06

(57) ABSTRACT

The present invention provides a personal lubricant composition that includes (a) at least one silicone-containing component, and (b) a hydrophilic component comprising water and a polyol in respective amounts, which provide advantages such as to tactile and sensory, feel.

### FIELD OF THE INVENTION

This invention is directed to personal lubricant compositions.

### BACKGROUND OF THE INVENTION

Personal lubricants for intimate contact are well known and are useful for providing lubricity to various parts of the body, e.g. mucous membranes (oral, rectal, vaginal). These compositions range from jellies to liquids to vaginal suppositories and vary from being aqueous to oils to silicone based.

Water-based personal lubricants that are water-soluble are the most widely-used personal lubricants. The viscosity of these products can be altered by adjusting their water content and concentration of cellulose or other gel-forming hydrophilic ingredients. Because water-based personal lubricants absorb into the skin and evaporate, most water-based lubricants have a tendency to dry out during use, but reapplication of the lubricant or application of water or saliva is usually sufficient to reactivate them. When the lubricant eventually dries out, it may leave behind a residue derived from the other ingredients in the formulation. In particular, lubricants containing sugar, glycerin, or other chemicals or preservatives create a sticky residue and associated sensation, sometimes along with a taste and smell that some may perceive as unpleasant. This may require reapplication during sex and/or removal of the residue with water.

On the other hand, silicone lubricants offer superior lubrication, but, can feel greasy, be difficult to clean up, and generally stain fabrics such as cotton, polyester or satin.

Silicone-based lubricants are usually formulated with fewer than four ingredients and do not contain any water, providing a different feel from water-based personal lubricants. Silicone-based lubricants are not absorbed by skin or mucus membranes, and consequently last longer than water-based lubricants. Because silicone lubricants do not wash off in water, they are perfect for play in the shower or other wet environments. Many different silicone lubricants are commercially available with varying quality and performance. Silicone-based lubricants are not usually recommended for use with sex toys or other products that are made from silicone because the formula may dissolve the surface making it sticky to the touch, and cause disintegration of the item over time

Lubricants have also been incorporated into coating of condoms to facilitate insertion of the penis into the vagina and to improve comfort during intercourse. The lubricant may also contribute to a reduction in the risk of failure of the condom by lowering the friction between the condom and the vaginal wall. Silicone-based lubricants have been used in the manufacture of pre-lubricated condoms due to long-lasting properties and superior latex compatibility of such lubricants.

Some condoms carry a coating of a water-based lubricant. Water-based lubricants offer some improvement in lubrication but also suffer additional disadvantages, such as causing the latex from which the condom is formed to hydrate and become white and opaque, which detracts from the aesthetic appearance of the condom.

U.S. 2010/0012132 discloses a multi-phasic lubricant composition, including new personal lubricants, new condom products, and new methods of making personal lubricants and condom products. The lubricant composition comprises at least two substantially immiscible phases comprising: a) a hydrophilic component, which may in certain embodiments comprise an aqueous phase, and in other embodiments lack substantial amounts of water; and b) a silicone component, such as a phase containing silicone oil, wherein the composition is substantially clear. As disclosed therein, the respective refractive indices of the silicone and hydrophilic components of the composition are made to match within =certain narrow limits so as to render them substantially undetectable on interface between the phases under ordinary lighting, or so as to render the composition clear and translucent.

Accordingly, there remains a need for a lubricant that combines the advantages of water-based and silicone-based formulations while minimizing their respective disadvantages.

### SUMMARY OF THE INVENTION

The present invention provides a personal lubricant composition that includes (a) at least one silicone-containing component, and (b) a hydrophilic component comprising water and a polyol in respective amounts, which provide advantages such as to tactile and sensory feel.

### BRIEF DESCRIPTION OF THE DRAWINGS

FIG. 1 through FIG. 8 are graphical representations of subject answers from a statistical study carried out to compare the tactile and sensory feel of personal lubricants within the scope of the present invention with commercial personal lubricants.

FIG. 9 is a three-way graphical representation of compositions of useful personal lubricants in which the optimal composition zone is set forth.

FIG. 10 is a three-way graphical representation of compositions of this invention as in FIG. 9, and which further includes known prior art compositions.

### DETAILED DESCRIPTION OF THE INVENTION

In one broad aspect of the present invention, the present lubricant compositions comprise a lubricant composition comprising a silicone component, water and an alkylene glycol component (such as, without limitation, a propylene, hexylene, or butylene glycol component). A small amount of an emulsifier may be included. This composition is formulated as a personal lubricant having smoothness, lubricity, and longevity in use that is vastly superior to purely aqueous-based lubricants.

The, present invention is drawn to new multi-phasic lubricant compositions, including new personal lubricants, new condom products, and new methods of making personal lubricants and condom products.

The present lubricant composition comprises at least two substantially immiscible phases comprising: a) a hydrophilic component, which comprises an aqueous phase and a alkylene glycol; and b) a hydrophobic silicone component, such as a phase containing silicone oil.

In general, the composition of the present invention will comprise from about 25 to 45 wt. % water, 40 to 60 wt. % alkylene glycol, and 10 to 30 wt. % of the silicone component. More particularly, the composition will comprise 29.5 to 36.0 wt. % water, 45 to 52 wt. % alkylene glycol, and 13.0 to 23.5 wt. % of the silicone component. Preferably, the alkylene glycol is propylene glycol.

The hydrophobic silicone component of the present compositions often comprises a dimethicone (trimethyl siloxane-terminated polydimethylsiloxane) and/or phenyltrimethicone component. Additional or alternative silicones may be present; these additional or alternative silicone components may include one or more of the following: a dimethiconol (hydroxy-terminated polydimethylsiloxane) component, a cyclopentylsiloxane component, a dimethicone/vinyl dimethicone cross-polymer component. For example, a silicone component may be a D5 cyclic or small linear dimethicone, disiloxane vinyl cross polymer.

For all of the present compositions a wide range of desired viscosities can be easily obtained; the mixture of immiscible silicone and hydrophilic components permits the making of formulations having a relatively low coefficient of friction, which are not tacky and which wash well from skin and fabric compared to strictly silicone lubricants. Hydrophilic and silicone components having a given molecular weight tend to be more viscous than those possessing the same subunits but having a lower molecular weight. Therefore, a composition of the present invention may easily be made to possess a desired viscosity.

Thus, the present compositions may be made to be in the form of a liquid or gel, depending upon the particular intended use thereof. The present compositions may be formulated as liquid gels, pourable gels or thick jellies or as pourable liquids. Preferably, the viscosities are in a range of about 50 cps or about 100 cps to less than about 500 cps or less than about 1000 cps or less than about 2000 cps. However, as noted above, viscosities in the present compositions may range from about 1000 cps to about 10,000 cps for gel, and from 60,000 cps to about 500,000 cps or more for the jellies. If desired, the present compositions may also be impregnated into fabrics or polymers.

In recent years it has become increasingly popular to add various functional ingredients to personal lubricant compositions. For example, a preservative component may be added to guard against microbial growth. In certain aspects of the invention, fragrances and/or flavorants may be added to the lubricant compositions to enhance to subjects' pleasure when using the lubricant. In general, the additional components wilt comprise from about 0.01% or less to at most about 1.0% by weight.

The present compositions may also be used as vehicles to deliver medication or other treatment agents to biomembranes, including, but not limited to, hormones, antimicrobials, antibacterials, antibiotics, non-steroidal anti-inflammatory agents, spermicides, immunodilators, anaesthetics, plant extracts, vitamins, corticosteroids or antifungal agents and the like and mixtures thereof.

A particularly useful addition are agents capable of enhancing pleasurable feelings during sexual activity and/or to heighten sexual arousal. Using condoms containing lubricants which include, one or more sensorial agents have also been proposed.

Personal lubricants comprising functional ingredients are designed to cause physiological or physical changes in the area to which they are applied. These actives range from ingredients that self-warm when, exposed to moisture, e.g. polyols; ingredients that act on nerve endings to simulate a perceived sensation such as warming, cooling and/or tingling; and ingredients that increase localized blood flow, e.g. vasodilators.

Lubricants that provide a perceived sensation include, for example, Trojan® Fire & Ice® condom that utilizes vanillyl butyl ether (VBE) to simulate a perceived warming sensation and menthol to simulate a perceived cooling sensation. Chemical warming agents (such as VBE) bind to the transient receptor potential type V1 channel (Vanilloid receptor-1, TRPV1) on sensory neurons. Chemical cooling agents (such as menthol) bind to TRP melastatin 8 (TRPM8) or cold and menthol receptor 1 (CMR1).

### Warming Agents

As used herein, warming agents refer to compounds that impart heating or a warming sensation upon topical application to the skin, oral cavity, throat or mucous membrane. Warming agents may be selected from the group consisting of capsaicin, gingerol, vanillyl ethyl:ether, vanillyl propyl ether, vanillyl butyl ether, vanillylpentyl ether, vanillyl hexyl ether, vanillyl;. butyl, ether acetate, 4-(1-menthoxymethyl)-2-phenyl-1,3-dioxolan, 4-(1-menthoxymethyl)-2-(3′,4′-dihydroxyphenyl)-1,3-dioxolan, 4-(1-menthoxymethyl)-2-(2″-hydroxy-3′-methoxyphenyl)-1,3-dioxolan, 4-(1-menthoxymethyl)-2-(4′-methoxyphenyl)-1,3-dioxolan, 4-(1-menthoxymethyl)-2-(3′,4′-methylenedioxyphenyl)-1,3-dioxolan, 4-(1-mentoxymethyl)-2-(3′-methoxy-4′-hydroxyphenyl)-1,3-dioxolan, red pepper oil, red pepper oleoresin, ginger oleoresin, nonylic acid vanillyl amide, Spilanthes acmella extract, Zanthoxylum alatum extract, Zanthoxylum piperitum extract, sanshool I, sanshool II, sanshoamide, black pepper extract, chavicine, piperine, spilanthol, or those warming agents disclosed in U.S. Pat. No. 6,780,443, which is hereby incorporated by reference in its entirety. As will be appreciated by those skilled in the art, certain of these agents may well be deemed tingling agents as opposed to warming agents.

As would be understood by one of ordinary skill in the art, the same compound may act differently depending on its use level in the composition. For example, a compound may act as a warming agent at a certain use level, yet the same compound will act as a tingling sensate at a higher use level.

### Cooling Agents

When cooling agents (also known as cooling sensates) are added to compositions containing warming agents, the cooling agent increases the warming effect of the warming, agent. See, e.g. U.S. Pat. No. 6,780,443, which is hereby incorporated by reference. Accordingly, topical warming compositions of the present invention may contain a “cooling agent.” In embodiments in which a warming effect is desired, the compositions should contain higher amounts of warming agents than cooling agents.

It has also been found that a topical cooling composition containing a cooling, agent (and smaller amounts of or no warming agent) increases the cooling effect of the cooling agent. Therefore, the present invention also provides compositions that include a cooling agent contained in a silicone-containing fluid carrier. These compositions contain a majority of a cooling agent, based on the total amount of sensates added to the composition.

Cooling sensates which may be included in compositions of the present invention include, but are not limited to, menthol, menthone, camphor, pulegol, isopulegol, cineol, mint oil, peppermint oil, spearmint oil, eucalyptus oil, 3-1-menthoxypropane-1,2-diol, N-alkyl-p-methane-3-carboxamide, 3-1-menthoxy-2-methylpropane-1,2-diol, p-menthane-3,8-diol, 2-1-menthoxyethane-1-ol, 3-1-menthoxypropane-1-ol, 4-1-menthoxybutane-1-ol, 1-(2-hydroxy-4-ethylcyclohexyl)-ethanone, menthyl 3-hydroxybutanoate, menthyl lactate, menthone glycerin ketal, 2-(2-1-menthyloxyethyl)ethanol, menthyl glyoxylate, N-methyl-2,2-isopropylmethyl-3-methylbutanamide, menthyl 2-pyrrolidone-5-carboxylate, monomenthyl succinate, alkali metal salts of monomenthyl succinate, and alkali earth metal salts of monomenthyl succinate, monomenthyl glutarate, alkali metal salts of monomenthyl glutarate, alkali earth metal salts of monomenthyl glutarate, N-[[5-methyl-2-(1-methylethyl)cyclohexyl]carbonyl]glycine, p-menthane-3-carboxylic acid glycerol ester, Menthol propylene glycol carbonate; Menthol ethylene glycol carbonate, and 6-isopropyl-3,9-dimethyl-1 ,4-dioxaspiro[4,5]decan-2-one.

Other cooling senates are disclosed in U.S Pat. Nos. 7,030,273 and 6,780,443, which are hereby incorporated by reference in their entirety.

For example, a compound may act as a cooling agent at a certain use level, yet the same compound will act as a tingling sensorial agent at a higher use level In particular, this is, true for menthol and menthol derivatives.

In other embodiments of the present invention, sensates besides warming agents and cooling agents are added in order to enhance the sensate material. For example, in one embodiment of the present invention a tingling sensate is added to enhance the tingling effect of the sensate material.

In general, one or more sensorial agents can be added to the compositions of this invention. Typically, the total amount of sensorial agents will range from 0.005 to less than 1.0 wt. %.

### EXAMPLE 1

### Home Use Test

The purpose of this test was to evaluate and compare the sensory characteristics of four personal lubricant products.

### Materials & Method

Three monadic home-use studies were conducted under identical conditions. Each study contained five to six cells of up to 150 subjects per cell for a total of 17 lubricants tested. Each cell was assigned a different lubricant to use at home, and then subjects were all given the same questionnaire to fill out. Subjects were blinded as to which lubricant they received.

Some of the lubricants included in the study were currently marketed products, while others were test formulas that comprised silicone-glycol-water hybrid-based formulas.

For the purposes of this analysis, two silicone-glycol-water hybrid lubricants (Test Lubricant #1 and Test Lubricant #2) were chosen to compare to a currently marketed silicone lubricant (K-Y® Intrigue®) and a currently marketed glycol lubricant (K-Y® Liquid ‘Natural Feeling’). The data for these four lubricants were split from the full data, and then each quantitative question from the questionnaire was analyzed to determine if the hybrid lubricants received results on questions related to overall-liking, rheology, skin-feel and convenience, which were distinct from the silicone lubricant and the glycol lubricant. Table 1 sets forth the composition of the test lubricants.

TABLE 1

| Product | | % w/w | | |
|---|---|---|---|---|
| No. | Description | Emulsil ® WSL | Water | Prop. Glycol |
| #1 | Hybrid Lubricant | 15.00 | 32.50 | 52.50 |
| #2 | Hybrid Lubricant | 17.00 | 31.00 | 52.00 |

*Emulsil ® WSL a pre-emulsified silicone compound from ChemSil Silicones, Inc.

### Results:

For each question, an Analysis of Covariance (ANCOVA) model was fit which included the factors of gender and lubricant, the interaction between gender and lubricant, and the subject's age as a covariate. For some questions all terms were significant, while for others some of the terms were not significant. Non-significant terms were removed from each model.

These questions were classified into one of three categories based on the type of statistically significant results observed.
Category 1: Questions wherein no significant differences were detected.
Category 2: Questions displaying significant differences between lubricants.
Category 3: Questions wherein the differences between lubricants were not statistically significant, but differences between the male and female responses created significant interactions between gender and lubricant.

Comparisons between the four lubricant means (for questions in, category 2) and between the eight gender-lubricant means (for questions in category 3) utilized post-ANCOVA t-tests. Tukey's method was used to control for multiple comparisons.

### Category 1 Questions:

Participants were asked to rate the overall-liking of the four lubricant products on a scale of 1 to 7, where a 1 is dislike and 7 is like. There was no statistical significance detected among the products. The products were equally liked.

Participants were asked whether they would agree or disagree, on a scale of 1-disagree to 5-agree, with the statement that the tested product is “a long lasting lubricant.” There was no statistical significance among the products in regards to this question.

The following categories below represent questions wherein the two hybrid lubricants (#1 and #2) received responses which were distinct from the silicone lubricant (K-Y® Intrigue®) and glycol lubricant (K-Y® Liquid ‘Natural Feeling’).

### Category 2 Questions:

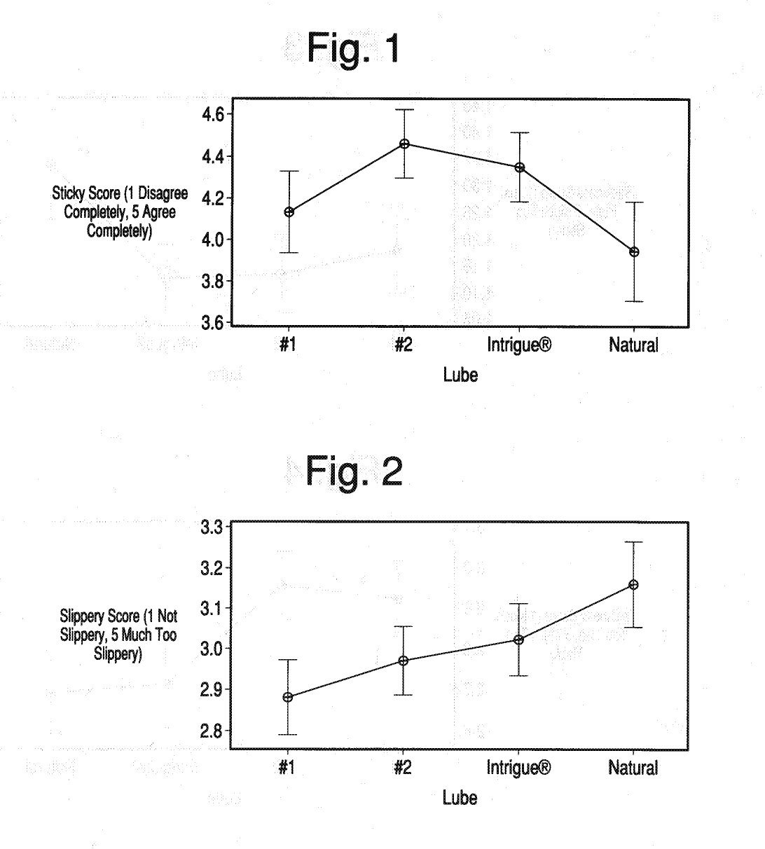

FIG. 1: Do you agree: “The lubricant is not sticky”? (1-Disagree to 5-Agree)
#2 and KY-Intrigue were statistically significantly higher than KY Natural Feeling.
#2 Natural Feeling P-value 0.0013

### Intrigue-Natural Feeling P-value 0.0196

FIG. 2: How did the lubricant feel? (1-Not slippery to 5-Too slippery with 3-About right)
KY Natural Feeling was statistically significantly higher than #2 and #1.
KY Natural #2 P-value 0.0119
KY Natural #1 P-value 0.0002

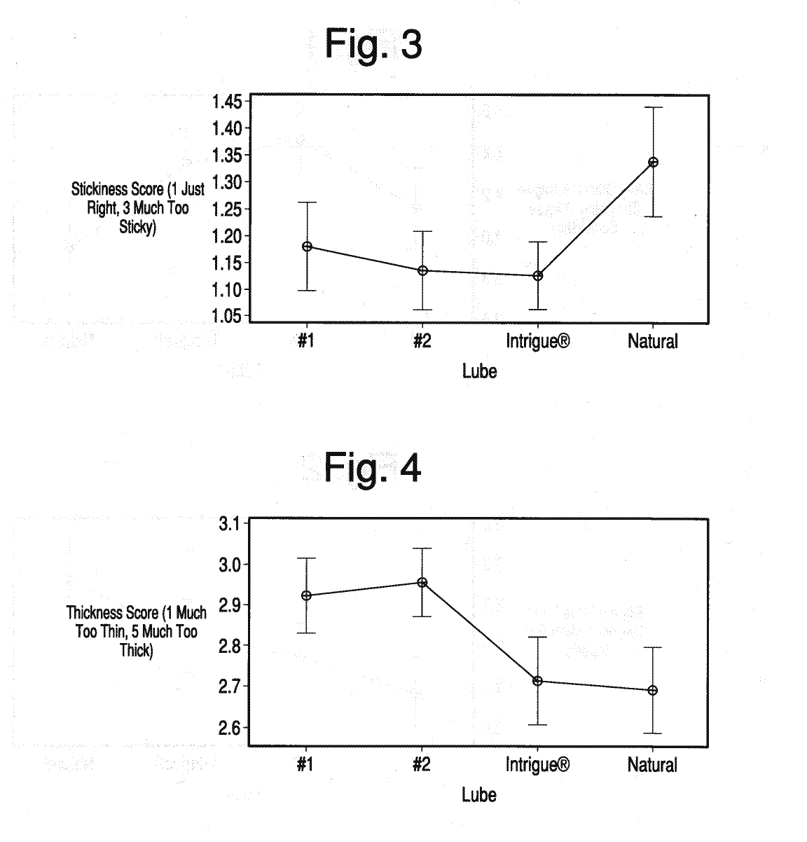

FIG. 3: How did the consistency of the lubricant feel? (1-About right to 3-Too sticky)
KY Natural Feeling was statistically significantly higher than #2 and #1, and KY-Intrigue
#1 Natural Feeling P-value 0.0276
#2 Natural Feeling P-value 0.0043
Intrigue/Natural Feeling P-value 0.0013

FIG. 4: Was the lubricant too thin or thick? (1-Too thin to 5-Too thick with 3-About right)
#1 and #2 were statistically significantly higher than both KY Intrigue and KY Natural Feeling
#2/KY Intrigue P-value 0.0044
#2/KY Natural Feeling P-value 0.0014
#1/KY Intrigue P-value 0.0289
#1/Natural Feeling P-value 0.0113

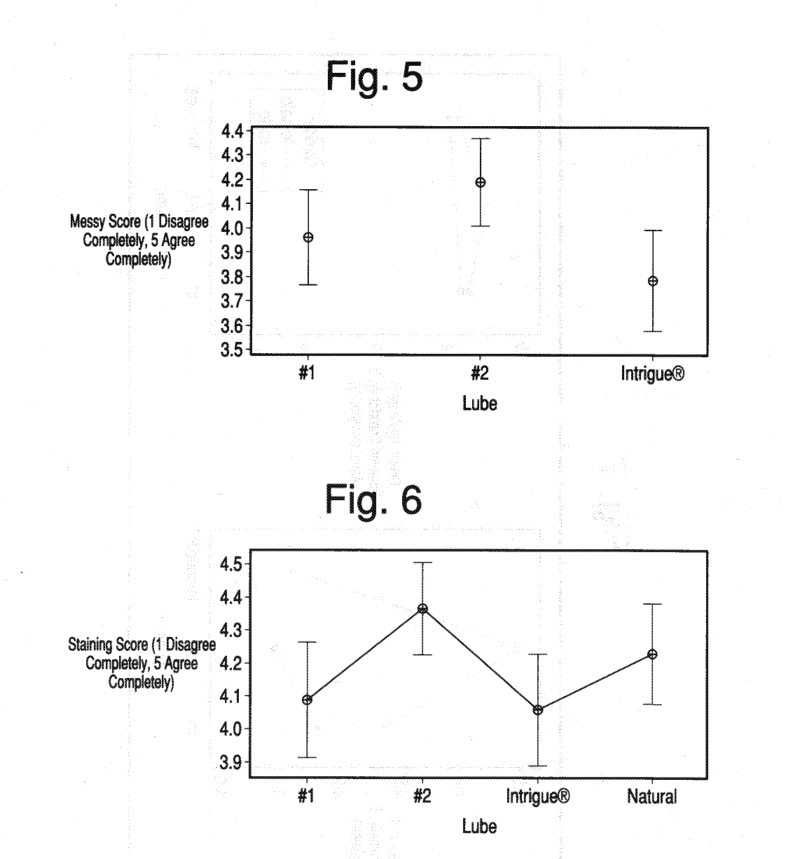

FIG. 5: Do you agree: “The lubricant is not messy”? (1-Disagree to 5-Agree)

#2 was statistically significantly higher than KY-Intrigue with a P-value of 0.0085

FIG. 6: Do you agree: “The lubricant does not stain bed sheets”? (1-Disagree to 5-Agree)
#2 was statistically significantly higher than KY Intrigue with a P-value of 0.0368
Category 3 Questions:

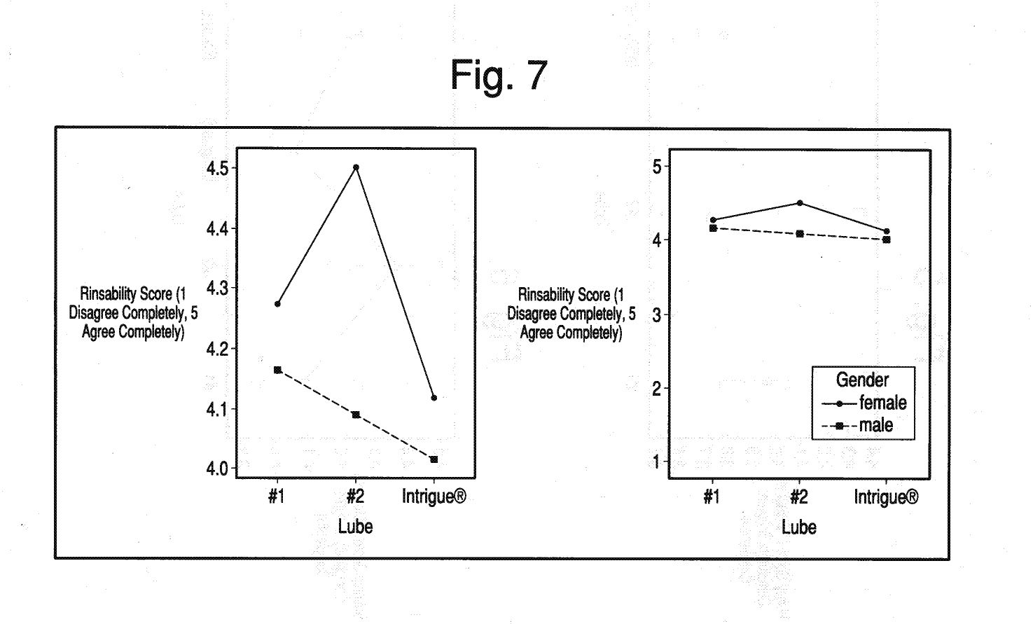

FIG. 7: “Easy to rinse from skin after use”? (1-Disagree to 5-Agree) #2 Female use was directionally higher than Intrigue® Male use with a P-value of 0.0648

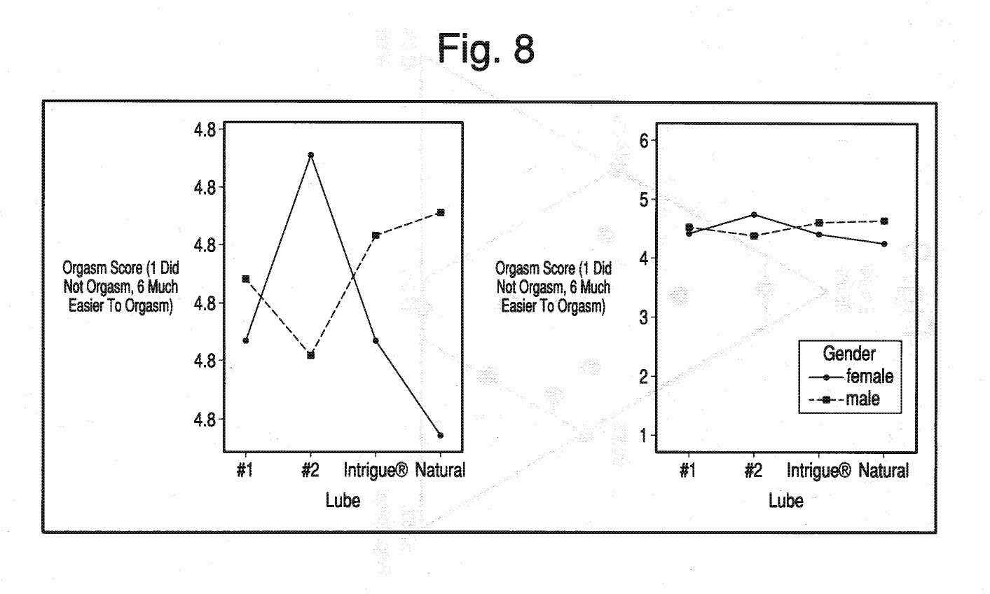

FIG. 8: How did your orgasm compare to the usual? (2-More difficult to 6-Easier with 1-No orgasm)

#2 Female use was statistically significantly higher than Natural Feeling Female use with a P-value of 0.0282

### EXAMPLE 2

In this study, members of an expert test panel utilized standard descriptive analysis testing methods (Sensory Evaluation Techniques, 3rd Edition 1999) to provide sensory data for product feel and appearance. Products were presented in a sequential monadic format, and all panelists saw all products in sequential monadic design. No more than two replicated samples were evaluated in a single session.

Each visual evaluation was performed using approximately 1 teaspoon of product. Each product was dispensed onto the forearm of a single subject for evaluation among the panelists. Panelists evaluated the sample for integrity of shape. Appearance evaluation took place using fluorescent and incandescent light.

For tactile evaluations, panelists washed their arms with low residue soap prior to evaluation and did not apply lotions. Each tactile evaluation was performed using approximately ½ teaspoon of product. Each product was dispensed onto the inner forearm near the crook. The product was spread in a gentle circular motion using the index finger. At measured time points, each sample was measured for the attributes listed in Table 2.

TABLE 2
The feeling upon application (first 15 seconds)

| | |
|---|---|
| Wetness | Amount of water perceived while rubbing. |
| | [none - - - high amount] |

The immediate after feel and 5 min after application

| | |
|---|---|
| Stickiness | Degree to which fingers adhere to product on skin. |
| | [not sticky - - - very sticky] |
| Slipperiness | Ease of moving fingers across skin. |
| | [difficult/drag - - - easy slip] |
| Oily | % designation of perceived residue. |
| | [none - - - large amount] |
| Greasy | % designation of perceived residue. |
| | [none - - - large amount] |

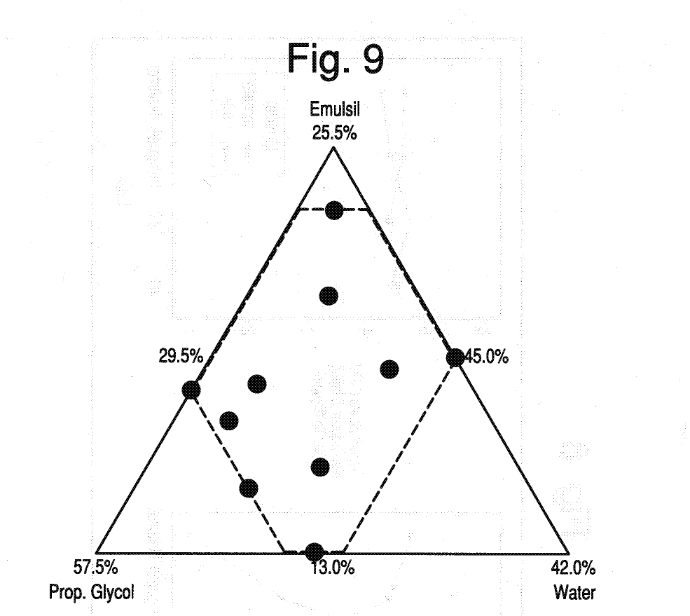

From the analysis of compositions containing varying amounts of silicone, propylene glycol and water, a zone was formed where all five characteristics as noted above were expected to simultaneously produce similar results. The zone included silicone ranging from 13.0% to 23.5% of the mixture, while propylene glycol ranged from 45% to 52% of the mixture and water ranged from 29.5% to 36.0% of the mixture. The formulations contained within the optimal zone are shown in Table 3 and depicted in FIG. 9.

TABLE 3
% w/w

| Emulsil | Water | Prop. Glycol |
|---|---|---|
| 15.00 | 32.50 | 52.50 |
| 17.00 | 31.00 | 52.00 |
| 19.00 | 36.00 | 45.00 |
| 18.00 | 29.50 | 52.50 |
| 23.50 | 30.50 | 46.00 |
| 13.00 | 35.25 | 51.75 |
| 18.67 | 34.42 | 46.92 |
| 18.17 | 31.17 | 50.67 |
| 20.92 | 31.67 | 47.42 |
| 15.67 | 34.04 | 50.29 |

### Table 3 and FIG. 9

The triangular space of FIG. 9 represents the potential combination of ingredient percentages in the formula. For each ingredient, the potential percentage of the formula ranges from a high at the corner with the ingredient label to a low at the side opposite that corner. (For example, Propylene Glycol ranges from a high of 57.5% of the formula to a low of 45.0% of the formula.)

The dashed line represents the boundary of the design space for the optimized formulas. That is, the two triangles in the bottom corners of the graph are outside the design space. The dots represent one of the formulas of Table 3 chosen to create a grid within the optimal design space.

### EXAMPLE 3

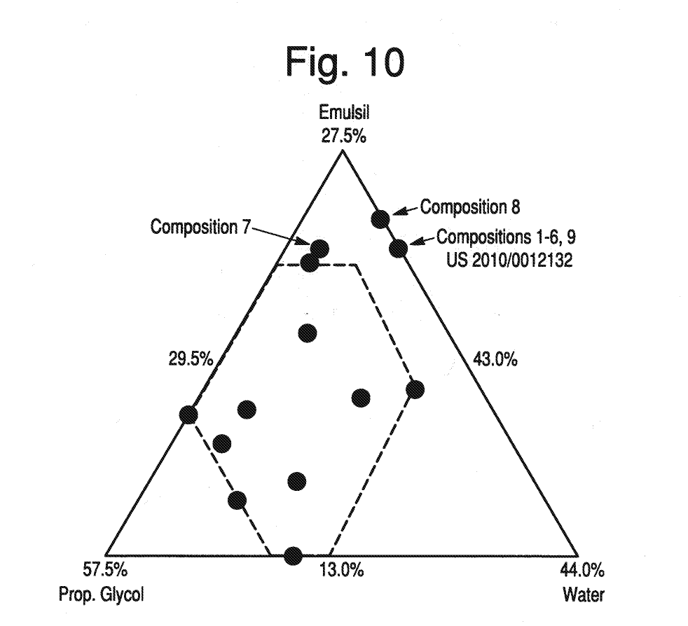

The optimized formulas as set forth in Table 3 were graphed as in FIG. 9, and plotted along with compositions 1-9 from U.S. 2010/0012132 as shown in FIG. 10. In FIG. 10, the propylene glycol ranges from a high of 57.5% of the formula to a low of 43.0%, so as to accommodate the formulations of the mentioned U.S. publication. FIG. 10 also shows a water content from a low of 29.5% to a high of 44.0%. The propylene glycol content of the formulations of the comparable U.S. publication was determined by adding all components, other than the silicone and water. Thus, the comparable formulations may include other glycols and/or preservative agents. It can be seen that the comparable formulations are all outside the space of the optimized formulations of the present invention.

1. A personal lubricant composition comprising: a) a hydrophilic component which comprises an aqueous phase and an alkylene glycol; and b) a hydrophobic silicone component, said composition comprising 29.5-36.0 wt. % water, 45-52 wt. % alkylene glycol and 13.0-23.5 wt. % of the silicone component.

2. The personal lubricant composition of claim 1, wherein the amounts of each of said water, alkylene glycol and silicone component fall within the zone defined by the dashed lines in FIG. 9.

3. The personal lubricant composition of claim 1, wherein the alkylene glycol is propylene glycol.

4. The personal lubricant composition of claim 2, wherein the alkylene glycol is propylene glycol.

5. The personal lubricant composition of claim 1, wherein the silicone component comprises a polymeric silicone component.

6. The personal lubricant composition of claim 5, wherein polymeric silicone component comprises at least one member of the, group consisting of a dimethicone component, a phenyltrimethicone component, a dimethiconol component, a cyclopentylsiloxane component and a vinyl dimethicone component.

7. The personal lubricant composition of claim 6, wherein said at least one polymeric silicone component is contained in a cross polymer.

8. The personal lubricant composition of claim 1, wherein the composition is pourable.

9. The personal lubricant composition of claim 1, wherein the composition is a gel form.

10. The personal lubricant composition of claim 1, having a viscosity in the range from about 50 cps to about 10,000 cps.

11. The personal lubricant composition of claim 10, wherein the amounts of each of said water, alkylene glycol and silicone component fall within the zone defined by the dashed lines in FIG. 9.

12. The personal lubricant composition of claim 2, wherein the silicone component comprises a polymeric silicone component.

13. The personal lubricant composition of claim 12, wherein polymeric silicone component comprises at least one member of the group consisting of a dimethicone component, a phenyltrimethicone component, a dimethiconol component, a cyclopentilsiloxane component and a vinyl dimethicone component.

14. The personal lubricant composition of claim 13, wherein said at least one polymeric silicone component is contained in a cross polymer.

15. The personal lubricant composition of claim 2, wherein the composition is pourable.

16. The personal lubricant composition of claim 2, wherein the composition is
